(19) United States
(12) Patent Application Publication
(10) Pub. No.: US 2009/0204027 A1
(43) Pub. Date: Aug. 13, 2009

(54) LANCET WITH SHIELDED LANCE

(76) Inventors: Robert F. Zuk, Atherton, CA (US); Hong TAN, San Jose, CA (US)

Correspondence Address: HOWREY LLP-CA, C/O IP DOCKETING DEPARTMENT, 2941 FAIRVIEW PARK DRIVE, SUITE 200, FALLS CHURCH, VA, 22042-2924, US

(21) Appl. No.: 12/369,521

(22) Filed: Feb. 11, 2009

Related U.S. Application Data
(60) Provisional application No. 61/027,737, filed on Feb. 11, 2008.

(51) Int. Cl. A61B 5/151; A61B 17/34

(52) U.S. Cl. 600/583; 606/181; 606/182

(57) ABSTRACT

An improved lancet as well as methods and devices for using the improved lancet are provided. The improved lancet comprises a body section with a lance arm and one or more protective arms extending from the body section. The lance arm has a lance with a sharp portion for piercing skin disposed on the lance arm. The sharp portion of the lance extends at an angle of about 45 to 135 degrees to the direction of extension of the lance arm from the body section. The protective arms have a protective section for shielding the sharp portion of the lance. The sharp portion of the lance is exposed from the protective section by application of pressure to the lance arm.

This application claims the benefit of U.S. Provisional Application No. 61/027,737, filed Feb. 11, 2008, which is incorporated herein by reference in its entirety.

### FIELD OF THE INVENTION

The present invention relates generally to systems and methods for testing body fluids. Particularly, the present invention relates to a lancet used for obtaining a sample of body fluid for testing that is simple, convenient and inexpensive. The present invention also relates to devices and methods for using the improved lancets.

### BACKGROUND OF THE INVENTION

With the rise in the incidence of certain infectious diseases, abused drugs or metabolic disorders, point of care (POC) or home testing is becoming more prevalent. Analytical tests designed for application in such environments must meet elevated requirements for safety, performance, simplicity, convenience and cost. In general, the state of the art in analytical chemistry has progressed to the point where such tests can achieve the performance requirements in terms of accuracy, precision and assay time. However, there is a continuing need to further improve the safety, simplicity, convenience and cost of POC tests.

Safety, simplicity, convenience and cost are barriers to adoption and regular use of current POC tests by consumers. Irregular use is a considerable problem for various chronic diseases where testing at regular intervals is mandatory. Tests perceived as inconvenient, too complex or too costly by the consumer are not suitable for use at the prescribed frequency. Consequently, the disease may not be effectively managed.

Glucose testing to manage Type II diabetes is one example of this problem. The glucose test itself takes a matter of seconds with highly accurate and precise results. However, from the consumer's point of view, the actual glucose test is only a portion of all the steps that must be taken to obtain a glucose level. Steps in a typical protocol for performing a glucose test are as follows:

1. Insert test strip in glucose meter

2. Insert lancet in lancing device

3. Remove protective cover from lancet

4. Lance skin

5. Contact blood with test strip

6. Read test result

7. Remove test strip from meter

8. Replace protective cover on lancet

9. Remove lancet from lancing device

10. Dispose used test strip

11. Dispose used lancet

The large number of steps makes the test less convenient for consumers and leads to reduced testing and ineffective management of Type II diabetes.

Another consideration in routine POC testing is the number of components required to run tests and their portability. In the case of monitoring glucose levels, the typical components include a container with multiple test strips, a container with multiple lancets, a lancing device and a glucose test meter. Although modern glucose meters can be pocket sized, carrying all of the necessary components together may still require a large pouch. As well as being inconvenient to carry, all of these components may also be expensive in the aggregate.

A further cost consideration in routine POC testing results from the number of lancets that must be purchased. As already discussed, POC testing may require sampling at routine intervals. Therefore, consumers often must purchase a significant number of lancets in order to comply with prescribed monitoring of their condition. Many of the prior art lancets and blood-testing devices sacrifice cost in the pursuit of convenience. These devices may provide convenience to the consumer but are prohibitively expensive so that consumers do not purchase the devices or do not purchase enough of the devices to accommodate their prescribed testing schedule.

For safety, many lancets have a protective cap to shield the lance from inadvertent contact. The protective cap must be removed prior to use and replaced after use. If the cap is not replaced, there is a danger of inadvertent contact with the contaminated lance resulting in a possible exposure to blood-born pathogens. A lancet that automatically shields the lance is advantageous because it improves convenience because the user does not have to remove or replace a cap. A lancet that automatically shields the lance is also advantageous because it reduces the risk of accidental contact with the lance. Unfortunately, devices heretofore designed to automatically shield the lance are cumbersome and expensive.

### SUMMARY OF THE INVENTION

An improved lancet as well as methods and devices for using the improved lancet are provided. The lancet comprises a body section with a lance arm and one or more protective arms extending from the body section. The lance arm has a lance with a sharp portion for piercing skin on the lance arm. The sharp portion of the lance extends at an angle of about 45 to 135 degrees to the direction of extension of the lance arm from the body section. The protective arms have a protective section for shielding the sharp portion of the lance. The sharp portion of the lance may be exposed from the protective section by application of pressure to the lance arm.

The blood sampling device of the present invention comprises a lancet as described and a housing with an insertion port for inserting the lancet. The housing also has a lancing button for depressing with a fingertip or alternative sampling site and an actuator. Depression of the lancing button causes the actuator to elevate the lance arm and lance of the lancet so that the sharp portion of the lance extends through the lancing button for piercing the skin of the fingertip or alternative sampling site used to depress the lancing button.

In an alternative blood sampling device of the present invention, the housing is elongated for holding in a closed hand. The housing has a lancing button for depressing with an appendage and an insertion port for insertion of the lancet at one end of the elongated housing. The housing also has an actuator. Depression of the lancing button causes the actuator to elevate the lance arm and lance of the lancet such that the sharp portion of the lance extends through the lancing button for piercing the skin of the appendage used to depress the lancing button.

Methods for sampling blood using the blood sampling device of the present invention are also provided. In the methods, the blood sampling devices described above are provided, the lance arm is raised to expose the sharp portion of the lance from protective section such that the lance pierces the skin to yield blood, blood is transferred to the test strip of the lancet, and the test strip is analyzed without removing the lancet from the blood sampling device.

### BRIEF DESCRIPTION OF TILE DRAWINGS

FIG. 1 is a top-down illustration of an embodiment of the lancet of the present invention with one protective arm.

FIG. 2a is a top-down illustration of an alterative embodiment of the lancet of the present invention with one protective arm.

FIG. 2b is a side view of the embodiment of FIG. 2a.

FIG. 2c is a side view of the embodiment of FIG. 2a showing displacement of the lance arm and lance.

FIG. 3a is a top-down illustration of an alternative embodiment of the lancet of the present invention with one protective arm.

FIG. 3b is a bottom-up view of the embodiment of FIG. 3a.

FIG. 3c is a side view of the embodiment of FIG. 3a.

FIG. 3d is a side view of the embodiment of FIG. 3a showing displacement of the lance arm and lance.

FIG. 4 is a top-down illustration of an embodiment of the lancet of the present invention with two protective arms.

FIG. 5a is a side view of the embodiment of FIG. 4 showing displacement of the lance arm and lance.

FIG. 5b is an end view of the embodiment of FIG. 4 showing displacement of the lance arm and lance.

FIG. 6a is a side view of the embodiment of FIG. 4 while the lance arm and lance are not displaced.

FIG. 6b is an end view of the embodiment of FIG. 4 while the lance arm and lance are not displaced.

FIG. 7 is a top-down illustration of as embodiment of the lancet of the present invention that also comprises a test strip.

FIG. 8a is a top-down illustration of an embodiment of the lancet of the present invention with illustrative dimensions.

FIG. 8b is a side view of the embodiment of FIG. 8a with illustrative dimensions.

FIG. 8c is a side view of the embodiment of FIG. 8a showing displacement of the lance arm and lance with illustrative dimensions.

FIG. 9a is an illustration of an embodiment of the blood sampling device of the present invention without a lancet inserted in the insertion port.

FIG. 9b is an illustration of an embodiment of the blood sampling device of the present invention with a lancet inserted in the insertion port.

FIG. 10a is a cross-section view of the embodiment of FIG. 9b with the lancet inserted in the insertion port.

FIG. 10b is a cross-section view of the embodiment of FIG. 9b with the lancet inserted in the insertion port showing displacement of the lance arm and lancet.

FIG. 11 is an illustration of an alternative embodiment of the blood sampling device of the present invention with a lancet inserted in the insertion port.

### DETAILED DESCRIPTION OF THE INVENTION

The lancet of the present invention has a shielded lance and is more simple, more convenient and less expensive than lancets in the prior art. The lancet of the present invention automatically shields the lance and can be inexpensively manufactured. The lancet of the present invention also minimizes the number of steps a user must follow and minimizes the number of devices a user must carry to sample blood.

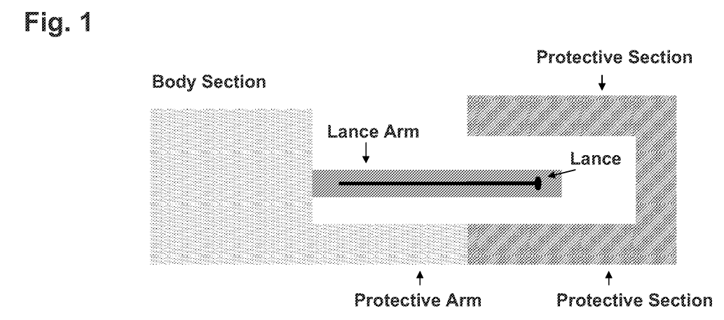

FIG. 1 shows a top-down view of an exemplary of an embodiment of the present invention. The lancet in FIG. 1 has a lance arm that extends from the body section. A lance is disposed on the lance arm. The lance may be any device capable of piercing the skin on a finger or other test area on the human body. The portion of the lance that is capable of piercing the skin is the sharp portion of the lance. The lance may be a needle, hook or the like and may be made of a plastic, metal or ceramic or the like. A protective arm also extends from the body section. A protective section is disposed on the protective arm. The protective section shields the sharp portion of the lance from contact when the lance is not in use. The protective arm extends from the body section so that the protective section shields the sharp portion of the lance. The protective arm is shown parallel to the lance arm in FIG. 1 but the protective arm may be at any angle as long as the protective section shields the sharp portion of the lance from contact when the lance is not in use.

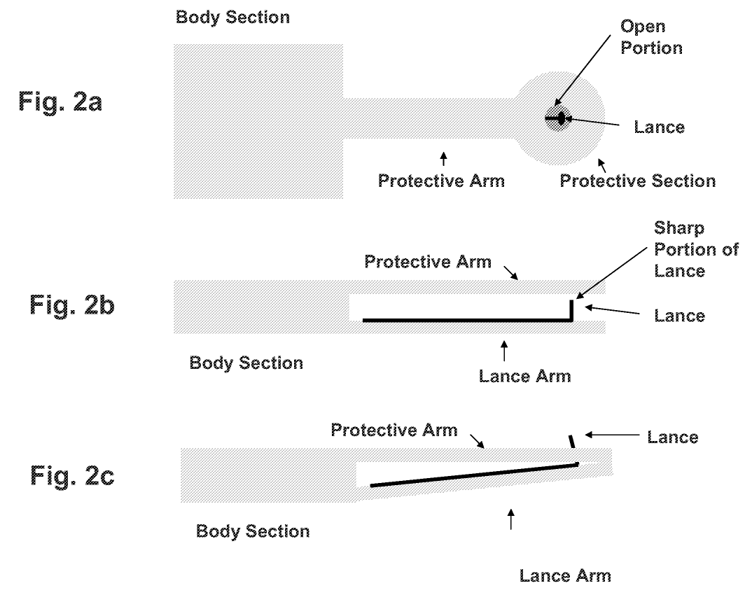

FIG. 2a shows a top-down view of an alternative embodiment of the present invention. In FIG. 2a, the protective arm extends from the body section above the lance arm. The protective arm has a protective section that shields the sharp portion of the lance from contact when the lance is not in use. In this embodiment, the protective section shields the sharp portion of the lance because the protective section is disposed above the lance and lance arm. The protective section has an open portion so that the sharp portion of the lance may be exposed to pierce skin when force is applied to the lance arm. FIG. 2b shows a side view of the lancet of FIG. 2a. FIG. 2b shows that the protective arm is disposed above the lance arm. FIG. 2c is also a side view of the lancet of FIG. 2a. FIG. 2c shows that the sharp portion of the lance is exposed to pierce skin when force is applied to the lance arm. In this embodiment, the sharp portion extends through the open portion of the protective section to be exposed for contact with skin.

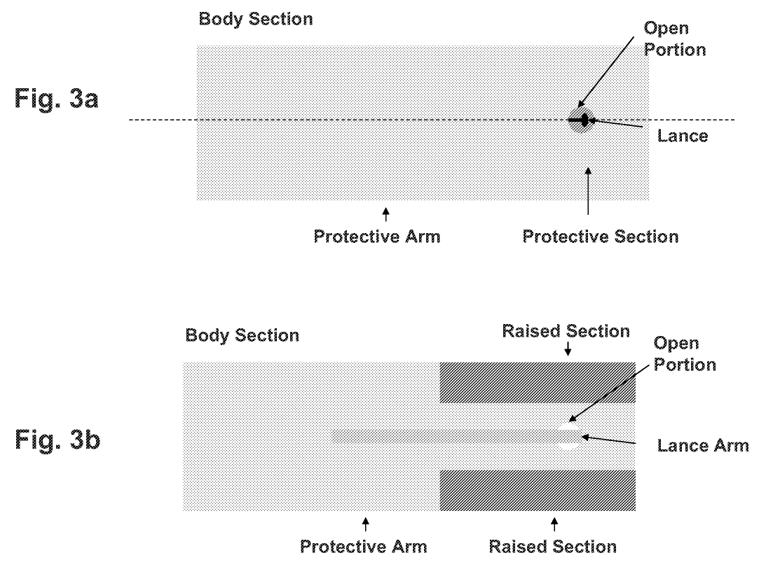

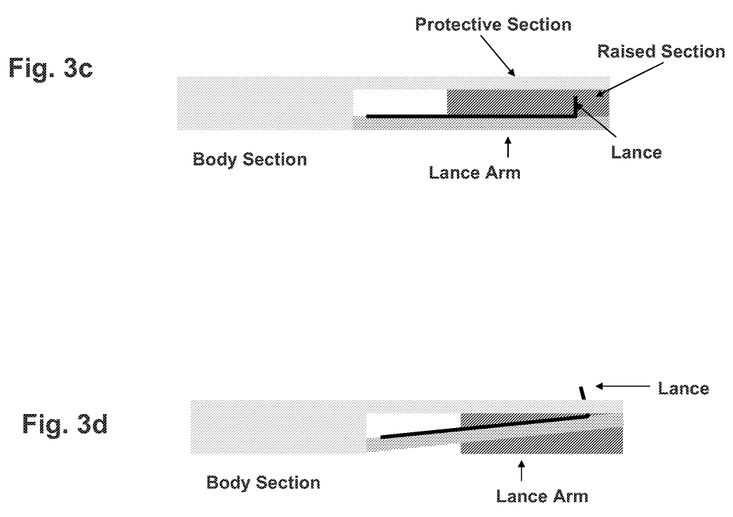

FIGS. 3a through 3d illustrate an alterative embodiment of the present invention. FIG. 3a shows a top-down view of this embodiment. FIG. 3b shows a bottom-up view of this embodiment. In this embodiment, the protective arm extends from the body section. As shown, the protective arm may be around the same width as the body section. Moreover, the protective arm may be integrally formed with the body section. That is, the body section and the protective arm may be formed in the same process step. For example, if both the protective arm and body are molded at the same time forming one cohesive unit. In this embodiment, the protective arm is disposed above the lance arm so that the protective section of the protective arm shields the sharp portion of the lance from contact when the lance is not in use. FIGS. 3c and 3d are cross-section views of the embodiment of FIG. 3a where the lancet of FIG. 3a is sectioned along the dotted line shown in FIG. 3a. FIG. 3c illustrates the relative locations of the lance arm and the protective arm above the lance arm. The protective section of the protective arm has an open section as shown in FIGS. 3a and 3b. FIG. 3d shows that the sharp portion of the lance may be exposed from the protective section by application of force on the lance arm. In this embodiment, the sharp portion is exposed to pierce skin by extension through the open portion of the protective section. As shown in FIGS. 3b, 3c and 3d, the lancet may also have one or more raised sections to prevent pressure from being applied to the lance arm when the lance is not in use.

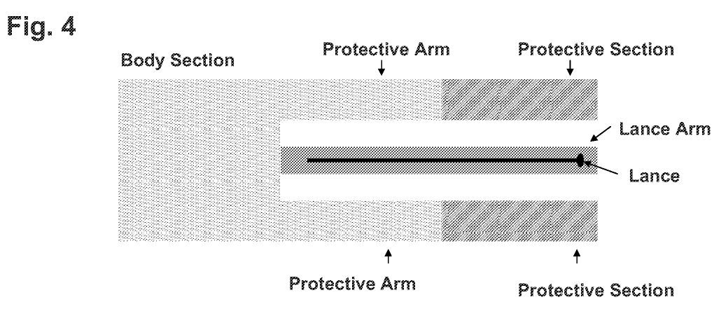

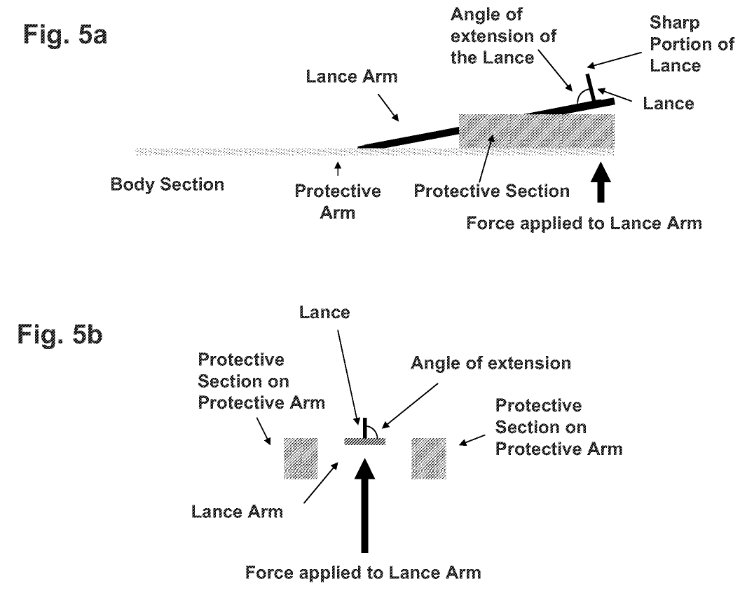

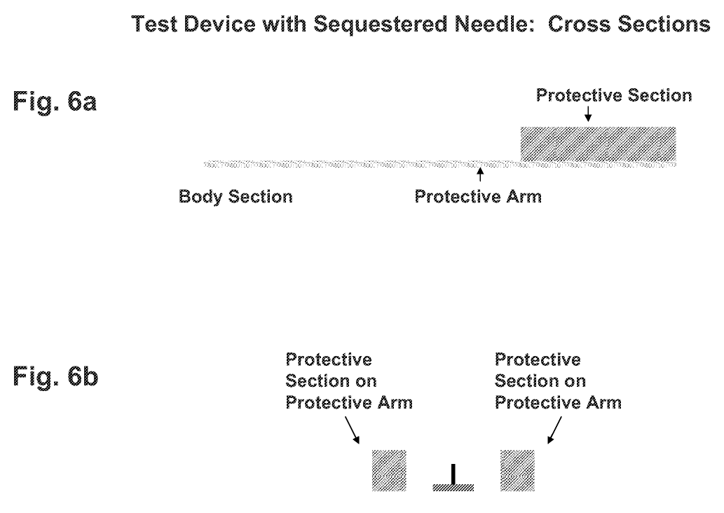

FIG. 4 shows a top-down view of an alternative embodiment of the present invention. In this embodiment, there are two protective arms that extend from the body section. On the protective arms, there is a protective section that shields the sharp portion of the lance when the lance is not in use. The lance is mounted, fixed, or integrally formed to a lance arm that extends from the body section. The sharp portion of the lance extends at an angle of about 45 to 135 degrees to the lance arm and at about 45 to 135 degrees from the protective arms. FIGS. 5a and 5b illustrate the orientation of the lance with respect to the lance arm and protective arms. FIG. 5a is a side view of this embodiment of the lancet with the sharp portion of the lancet exposed. FIG. 5a illustrates that the sharp portion of the lance extends at an angle about 45 to 135 degrees from the direction of extension of the lance arm from the body section. FIG. 5b is an end view of this embodiment of the lancet from the end opposite the body section. FIG. 5b shows that the sharp portion of the lance extends at an angle of about 45 to 135 degrees from the direction of the protective arms. FIGS. 5a and 5b show that when force is applied to the lance arm, the lance arm and lance move to expose the sharp portion of the lance. FIGS. 6a and 6b illustrate the orientation of the lance and lance arm of this embodiment when the lance is not in use. FIG. 6a is a side view of this embodiment of the lancet. As shown in FIG. 6a, the protective section shields the sharp portion of the lancet. FIG. 6b is an end view of the lancet taken from the end opposite the body section. FIG. 6b shows that when the lance is not in use, the protective sections extend above the sharp portion of the lance so that the sharp portion is shielded from contact.

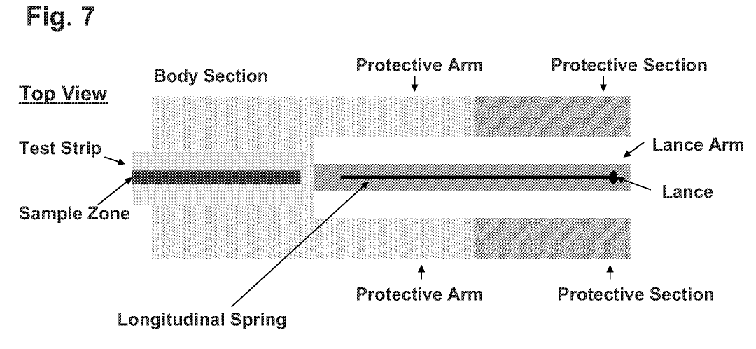

The lancets of the present invention may also comprise a test strip. The test strip may be adapted from a multitude of assays in a strip formats. Lateral flow assays or homogeneous assays in pads or channels may be used. Well-know methods of measuring or analyzing test strips are described in C. Price et al., Point of Care Testing (2nd ed. 2004), the entire disclosure of which in hereby incorporated by reference. As shown in FIG. 7, the test strip may be located on the body section of the lancet. The test strip may be adhered to the lancet. Methods of bonding the test strip to the lancet may include gluing, sonic welding, double stick adhesive or other similar means as well as any combination of these means. The test strip may be a conventional test strip that is widely available for use with other devices such as standard glucose test strips. The test strip may alternatively be specifically adapted for use with the lancet of this invention.

The body section, the lance arm and the protective arms of the lancet may be made of plastic. Suitable plastics include polystyrene, polypropylene, polycarbonate, polymethylmethacrylate, and the like. The body section, the lance arm and the protective arms may also be made out of paper such as cardboard or laminated paper or the like. Moreover, the body section, the lance arm, and the protective arms may be made from a combination of paper and plastic. The body section and any of the protective arms or the lance arm may be integrally formed. That is, the body section and the arms may be formed in the same process step. For example, the body section and one or more of the arms may be molded at the same time in one piece.

The lancet of this invention may also comprise a longitudinal spring as shown in FIG. 7. The longitudinal spring extends in the direction of extension of the lance arm. The longitudinal spring may be adhered to the lance arm. The longitudinal spring may be made of metal, plastic or some other material rigid enough to produce a spring effect. The longitudinal spring may be integrally formed with the lance. The longitudinal spring may be adhered to the body section or it may be embedded in the body section. The longitudinal spring may also be adhered to the lance arm. An example of an embedded longitudinal spring is one that is inserted into malleable plastic during molding. The longitudinal spring is at rest when the lance is not in use. FIG. 6b illustrates the position of the lance and lance arm when the lance is not in use. When force is applied to the bottom of the lance arm, as shown in FIG. 5b, the longitudinal spring is under tension and acts to return the lance arm to its original position as shown in FIG. 6b. The lance arm itself may be sufficiently rigid to return to its original position without the use of a longitudinal spring. The longitudinal spring may be used to make the lance arm return to its original position more quickly and/or more consistently than the lance arm alone. It is important for the lance arm to return to near its original position so that the sharp portion of the lance is shielded after use.

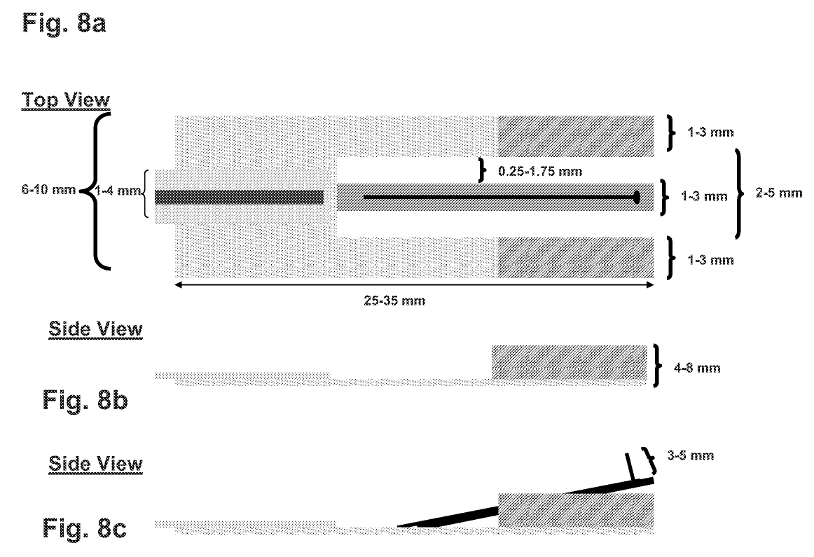

FIGS. 8a, 8b and 8c show a further embodiment of the present invention. In this embodiment, the combined length of the body section and protective arms is between about 25 and 35 mm. The width of the body section is between about 6 and 10 mm. The width of the protective arms is between about 1 and 3 mm. The width of the lance arm is between about 1 and 3 mm. The space in between the protective arms and the lance arm is between 0.25 and 1.75 mm. The width of the test strip is between about 1 and 4 mm. FIG. 8b shows a side view of this embodiment. As shown, the height of the protective section is between about 4 and 8 mm. FIG. 8c is a side view of this embodiment with the lance arm elevated to reveal the sharp portion of the lance. As shown, the height of the lance is between about 3 and 5 mm. The height of the lance from the top of the lance arm must be less than the height of the protective section from the top of the lance arm so that the sharp portion of the lance is shielded when the lance arm is not extended.

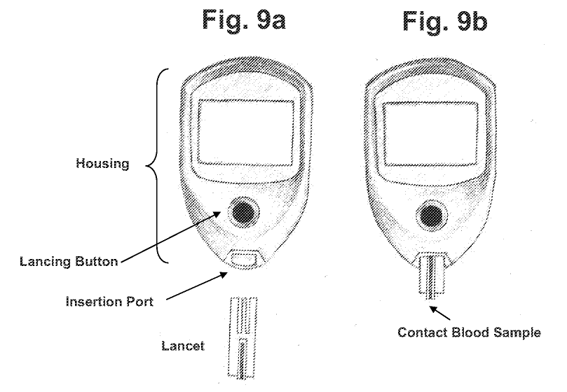

FIGS. 9a and 9b show a blood sampling device for use with the lancet of this invention. The device has a housing into which a lancing button and an insertion port are formed. The housing may be made of plastic, metal or similar materials or any combination of these materials. The insertion port is adapted to accept insertion of the lancet of this invention. FIG. 9a shows how the lancet may be inserted into the device. FIG. 9b shows the device with a lancet inserted. The device has a lancing button. The lancing button causes an actuator in the device to elevate the lance arm of the lancet when the lancing button is depressed. The actuator may be a spring-loaded piston or other device as known in the art capable of exerting force on the lance arm. The elevation of the lance arm causes the sharp portion of the lance to extend through the lancing button to pierce the skin of the finger or alternative sampling site used to depress the lancing button. Generally, the lancing button will be depressed by a finger, but in some situations an alternative sampling site on the body may be used. Therefore, the lancing button may be depressed by a finger or alternative sampling site. As shown in FIG. 9b, the test strip extends outside the insertion port. After the skin of the finger or alternative sampling site is pierced and blood is drawn, the blood is contacted with the exposed section of the test strip. The blood testing device may contain a means for analyzing the test strip after contact with the blood. The signal readout in the test strip could either be optical, fluorescence, chemiluminescence, or electrochemical

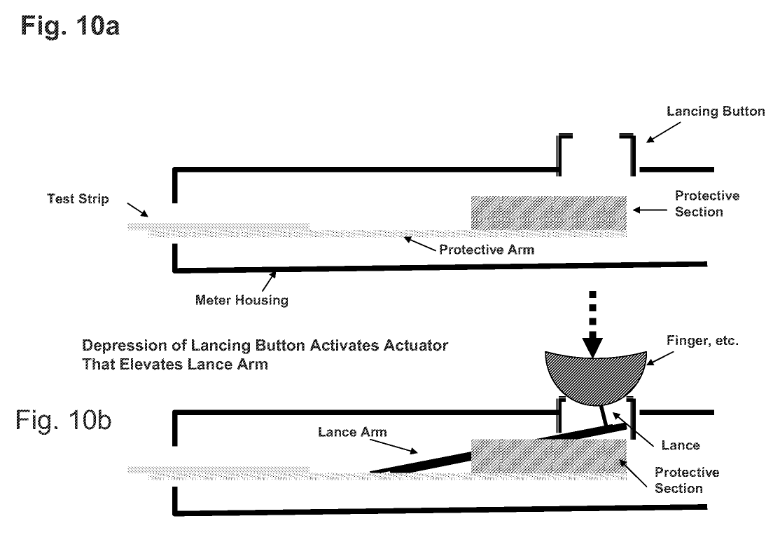

FIGS. 10a and 10b show a cross section side view of the blood testing device with a lancet of the present invention inserted. In FIG. 10a, the lance is not in use and the lancing button is not depressed. In FIG. 10b, the lancing button is depressed and the lance arm is elevated. As shown, the sharp portion of the lance is exposed through the lancing button to pierce the skin of the finger or alternative sampling site used to depress the lancing button.

The blood sampling device of the present invention allows for simpler testing since fewer steps are required to use this device than are required for devices in the art. To sample blood with the device of the present invention, the user only has to insert the lancet of this invention in to the insertion port and depress the lancing button. If the lancet includes a test strip, the user may then contact the blood to the test strip. If the blood sampling device includes a meter, the device reads the results from the test strip and displays them to the user.

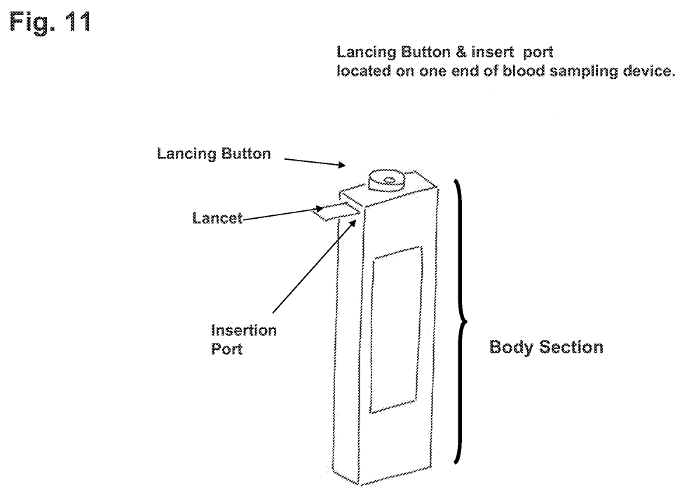

The housing of the blood sampling deice of this invention may also be elongated as shown in FIG. 11. This embodiment of the blood sampling device may be held in a closed hand so that the lancing button may easily be depressed by the thumb. This embodiment may also be used with an alternative test site by being held in the hand and depressing the lancing button on the alterative test site. This embodiment allows convenient one-hand operation of the blood sampling device. After the lancet is inserted in the insertion port, the user may hold the device in a closed hand, depress the lancing button with the thumb or alternative test site and then transfer blood to the test strip with the thumb or alternative test site. In a further embodiment, the device with an elongated housing may also comprise a means for analyzing the test strip without removing the test strip from the insertion port.

Using the blood sampling devices of the present invention, a user may sample blood by inserting the lancet into the insertion port, depressing the lancing button thereby raising the lance arm to expose the sharp portion of the lance from protective section such that the lance pierces the skin to yield blood, transferring the yielded blood to the test strip of the lancet, and analyzing the test strip without removing the lancet from the blood sampling device.

While the invention is susceptible to various modifications and alternative forms, specific embodiments have been shown by way of example in the drawings and detailed description herein. However, it should be understood that the invention is not intended to be limited to the particular forms disclosed. Rather, the intention is to cover all modifications, equivalents and alternatives falling within the spirit and scope of the invention as defined by the appended claims.

What is claimed:

1. A lancet comprising:
a body section;
a lance arm extending from the body section;
a lance having a sharp portion for piercing skin, wherein the lance is disposed on the lance arm and the sharp portion of the lance extends at an angle of about 45 to 135 degrees to the direction of extension of the lance arm from the body section;
a protective arm extending from the body section;
a protective section for shielding the sharp portion of the lance disposed on the protective arm, wherein the sharp portion of the lance is exposed from the protective section by application of pressure to the lance arm.

2. The lancet of claim 1, further comprising a test strip.

3. The lancet of claim 1, wherein the body section further comprises a test strip.

4. The lancet of claim 1, wherein the body section is made of plastic, paper, or the combination thereof.

5. The lancet of claim 1, wherein the protective arm is made of plastic, paper, or the combination thereof.

6. The lancet of claim 1, further comprising a longitudinal spring.

7. The lancet of claim 6, wherein the longitudinal spring comprises a metal.

8. The lancet of claim 6, wherein the lance and the longitudinal spring are integrally formed.

9. A blood sampling device comprising:
the lancet according to claim 1;
a housing;
an insertion port in the housing for inserting the lancet;
a lancing button for depressing with a fingertip or an alternative sampling site; and an actuator, wherein depression of the lancing button causes the actuator to elevate the lance arm and lance such that the sharp portion of the lance extends through the lancing button for piercing the skin of the fingertip or an alternative sampling site used to depress the lancing button.

10. The blood sampling device of claim 9, further comprising means for analyzing the test strip without removing the test strip from the insertion port.

11. A method for sampling blood comprising:
providing the blood sampling device of claim 9;
raising the lance arm to expose the sharp portion of the lance from protective section such that the lance pierces the skin to yield blood;
transferring the yielded blood to the test strip of the lancet; and
analyzing the test strip without removing the lancet from the blood sampling device.

12. A blood sampling device comprising:
the lancet according to claim 1;
an elongated housing for holding in a closed hand, wherein the elongated body has a proximal end and a distal end;
a lancing button for depressing with an appendage, disposed at the proximal end of the elongated housing;
an insertion port for insertion of the lancet, disposed at the proximal end of the elongated housing; and
an actuator, wherein depression of the lancing button causes the actuator to elevate the lance arm and lance such that the sharp portion of the lance extends through the lancing button for piercing the skin of the appendage used to depress the lancing button.

13. The blood sampling device of claim 12, further comprising means for analyzing the test strip without removing the test strip from the insertion port.

14. A method for sampling blood comprising:
providing the blood sampling device of claim 12;
raising the lance arm to expose the sharp portion of the lance from the protective section such that the lance pierces the skin to yield blood;
transferring the yielded blood to the test strip of the lancet; and
analyzing the test strip without removing the lancet from the blood sampling device.

15. The lancet of claim 2, wherein the body section is rectangular and is about 6 to 10 mm wide by about 10 to 14 mm long and about 1 to 3 mm thick;
The first and second protective arms extend about 16 to 20 mm from the body section and are about 1 to 3 mm wide and about 1 to 3 mm thick;
the lance arm extends about 16 to 20 mm from the body section and is about 1 to 3 mm wide and about 1 to 3 mm thick
the first and second protective sections rise about 4 to 8 mm from the at first and second protective arms, and the lance extends about 3 to 5 mm from the lance arm.

16. A lancet comprising:
a body section;
a lance arm extending from the body section;
a first protective arm extending from the body section;
a second protective arm extending from the body section;
a lance having a sharp portion for piercing skin, wherein the lance is disposed on the lance arm and the sharp portion of the lance extends at an angle of about 45 to 135 degrees to the direction of extension of the lance arm and at an angle of about 45 to 135 degrees to the first and second protective arms;
a first protective section for shielding the sharp portion of the lance from contact disposed on the first protective arm;
a second protective section for shielding the sharp portion of the lance from contact disposed on the second protective arm.

17. An blood sampling device comprising:
a housing;
an insertion port in the housing for inserting a lancet having a lance;
a lancing button for depressing with a fingertip or alternative sampling site; and
an actuator, wherein depression of the lancing button causes the actuator to elevate the lance such that the lance extends through the lancing button for piercing the skin of the fingertip or alternative sampling site used to depress the lancing button.

18. The blood sampling device of claim 17, further comprising means for analyzing the test strip without removing the test strip from the insertion port.

19. A blood sampling device comprising:
an elongated housing for holding in a closed hand, wherein the elongated body has a proximal end and a distal end;
a lancing button for depressing with an appendage, disposed at the proximal end of the elongated housing;
an insertion port for insertion of a lancet having a lance, disposed at the proximal end of the elongated housing; and
an actuator, wherein depression of the lancing button causes the actuator to elevate the lance such that the lance extends through the lancing button for piercing the skin of the appendage used to depress the lancing button.

20. The blood sampling device of claim 19, further comprising means for analyzing the test strip without removing the test strip from the insertion port.
